(19) United States
(12) Patent Application Publication
(10) Pub. No.: US 2008/0064982 A1
(43) Pub. Date: Mar. 13, 2008

(54) TISSUE SAMPLE PROTECTING CAUTERIZING BIOPSY FORCEPS

(76) Inventors: Brett NOWLIN, Bridgewater, MA (US); Ken Daignault, Holden, MA (US); Michael S. H. Chu, Brookline, MA (US)

Correspondence Address: FAY KAPLUN & MARCIN, LLP, 15O BROADWAY, SUITE 702, NEW YORK, NY, 10038, US

(21) Appl. No.: 11/763,586

(22) Filed: Jun. 15, 2007

Related U.S. Application Data
(60) Provisional application No. 60/843,963, filed on Sep. 12, 2006.

(51) Int. Cl. A61B 10/06

(52) U.S. Cl. 600/564

(57) ABSTRACT

Described are a method and biopsy device which includes a tissue cutting arrangement and a cauterizing element. The tissue cutting arrangement is operable to excise a tissue sample from a sampling site. The tissue cutting arrangement defines a sample chamber therewithin for receiving a tissue sample after excision by the tissue cutting arrangement. The sample chamber includes insulated sample contacting surfaces. The cauterizing element is formed on at least a portion of an exterior surface of the tissue cutting arrangement. The cauterizing element is actuatable to cauterize the sampling site after excision of the tissue sample.

### PRIORITY CLAIM

This application claims the priority to the U.S. Provisional Application Ser. No. 60/843,963, entitled “TISSUE SAMPLE PROTECTING CAUTERIZING BIOPSY FORCEPS,” filed Sep. 12, 2006. The specification of the above-identified application is incorporated herewith by reference.

### BACKGROUND INFORMATION

Biopsy is the preferred method of diagnosis for many diseases and conditions. When it is desired to biopsy tissue within certain body structures such as, for example, hollow organs or lumens, an endoscope or similar device may be used to reach target issue and to insert cutting and retrieval devices to that tissue via the working lumen of the scope.

Because of the cutting involved, cauterization elements are often inserted to the sampling site to stop the bleeding that may occur at the sampling site. These cauterization elements may be part of a separate device or may be included in the cutting and/or retrieval devices used to obtain the biopsy. However, it may be difficult with conventional devices to achieve a desired degree of cauterization (i.e., sufficient to stop the bleeding) without excessive damage to surrounding tissue. In addition, exposure to excessive heat may damage the tissue sample reducing or destroying its diagnostic value.

### SUMMARY OF INVENTION

The present invention relates to a biopsy device which includes a tissue cutting arrangement and a cauterizing element. The tissue cutting arrangement is operable to excise a tissue sample from a sampling site. The tissue cutting arrangement defines a sample chamber therewithin for receiving a tissue sample after excision by the tissue cutting arrangement. The sample chamber includes insulated sample contacting surfaces. The cauterizing element is formed on at least a portion of an exterior surface of the tissue cutting arrangement. The cauterizing element is actuatable to cauterize the sampling site after excision of the tissue sample.

The present invention also relates to a method of biopsy. An insulated biopsy forceps is advanced to a sampling site within a body lumen. A tissue sample from the sampling site is actuated from a cutting element of the forceps from outside the body to excise. The tissue sample is retained in a sample chamber within the forceps, the sample chamber having insulated interior surfaces. A cauterizing element is energized to cauterize the sampling site.

The present invention further relates to a biopsy system with cauterization. The system may include a shaft having a distal end insertable through a scope to a sampling site within a body; a non-conductive shaft coating covering at least a portion of the shaft; a housing disposed at a distal end of the shaft; a cutting element of the housing operable by a user to excise a sample tissue from the sampling site; a sample chamber defined within the housing for retaining the excised sample tissue; a cauterization element of the housing to cauterize the sampling site after excision of the sample tissue; and a non-conductive housing coating covering at least parts of surfaces of the housing.

### BRIEF DESCRIPTION OF DRAWINGS

FIG. 1 is a perspective view showing an embodiment of the biopsy forceps according to the invention in an open configuration;

FIG. 2 is perspective view of the biopsy forceps shown in FIG. 1 in the closed configuration;

FIG. 3 is a view of the biopsy forceps shown in FIG. 1 attached to a carrier device;

FIG. 4 is a perspective view of a biopsy forceps according to a second embodiment of the present invention, in a closed configuration;

FIG. 5 is a perspective view of the biopsy forceps shown in FIG. 4, in an open configuration;

FIG. 6 is a perspective view of a biopsy forceps according to a third embodiment of the present invention, with a separate cauterization element;

FIG. 7 is a perspective view of a biopsy forceps according to a different embodiment of the present invention, with a laser cauterization element; and

FIG. 8 is a perspective view of a biopsy forceps according another embodiment of the present invention, comprising a sample basket.

### DETAILED DESCRIPTION

The present invention may be further understood with reference to the following description and the appended drawings, wherein like elements are referred to with the same reference numerals. The present invention relates to devices for performing a biopsy. More specifically, the invention relates to devices used to retrieve biopsy tissue samples and to cauterize the resulting wound.

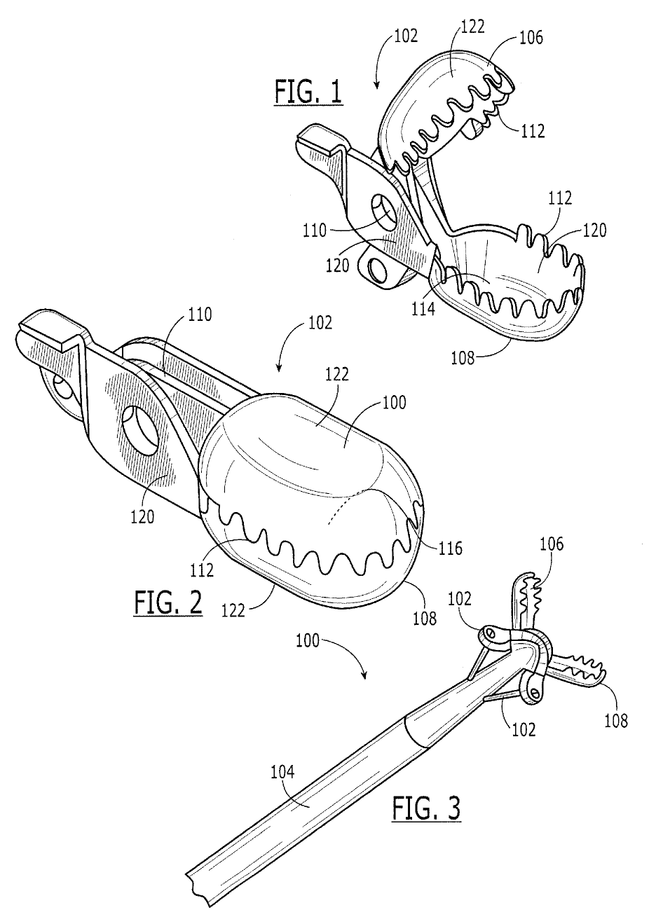

As shown in FIG. 1, a biopsy forceps 100 according to the present invention adapted to obtain a sample of tissue and immediately cauterize the sample site while preserving the tissue sampled therefrom comprises a shaft 104 used to advance through, for example, an endoscope the distal head 102 to the target sample area. The distal head 102 comprises the cutting instruments used to remove the sample tissue from its surroundings.

The forceps 100 according to the exemplary embodiment of the present invention comprises a pair of hinged jaws 106, 108 including overlapping teeth 112, or other similar cutting surfaces. The opposing cutting surfaces of the jaws 106, 108 may be brought together over the sample tissue once the distal head 102 has been advanced to the sampling site within, for example, a body lumen or cavity. The exemplary jaws 106, 108 pivot about a hinge 110, so that they can open and close using a remote actuator 104, that may be operated by a user from outside the patient's body.

The jaws 106, 108 are preferably shaped to form a sample holder, such as a sample cup 114 that becomes an enclosed sample chamber 116 once the jaws 106, 108 are closed. The sample chamber 116 retains the sample for removal from the body while protecting the sample from contamination during removal. The jaws 106, 108 are preferably designed to automatically enclose the sample within the chamber 116 after the sample has been excised to prevent loss of the sample during retrieval. Those of skill in the art will understand that different shapes and locations of the sample chamber 116 may be used, without departing the scope of the present invention.

Multiple surfaces of the biopsy forceps 100 are coated with an insulating material to prevent the non target tissue and the sample tissue from being damaged. For example, the insulation may comprise PTFE or PFA materials, ceramics or any other coating that reduces heat transfer and is electrically non-conductive. The insulating coating may be sprayed, dipped or painted on the device. As shown in the drawings, the device according to this embodiment comprises coated, non-conductive surfaces 120 formed as described above as well as non-coated, conductive surfaces 122 which deliver energy to cauterize the sampling site. A large portion of the exemplary device is coated with the non-conductive insulation, including the shaft 104 and the sample cup 114. As would be understood by those skilled in the art, for forceps according to alternate embodiments using other forms of energy (e.g., laser) to cauterize tissue, the insulation need not be electrically non-conductive and may be simply thermally insulative. For example, an electrically conductive, thermally insulative material such as conductive acetal polymer black carbon filled Pomaflux CN-F may be used to the shaft 104 and/or the sample cup 114. A laser may be used to heat the jaws 106, 108 for cauterizing tissue when need to be thermally insulated but not electrically insulated.

When the jaws 106, 108 are closed, the conductive portions 122 form a generally bullet shaped cauterization tool that may be used to stop bleeding within the sampling site. The user may, for example, visualize the site via an endoscope to direct the distal head 102 to the site of bleeding to cauterize and seal the wound. Because of the insulation disposed on the surfaces 120, the portion of tissue cauterized may be accurately targeted minimizing inadvertent cauterization of the surrounding tissue. The sample within the sample chamber 116 is also protected from damage during the cauterization steps by insulation coated on the inside portions of the jaws 106, 108.

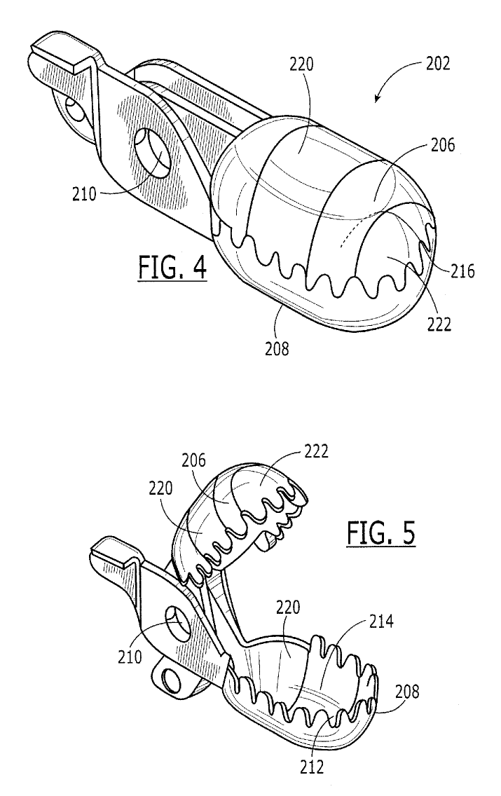

A second embodiment of a biopsy forceps according to the present invention is shown in FIGS. 4 and 5. In this embodiment the conductive region of the device is smaller and shaped to more closely direct RF energy path in a selected direction. As shown, the forceps 202 comprises a pair of jaws 206, 208 having a cutting portion 212 for removing tissue samples. A cup 214 is preferably formed by the jaws 206, 208 to hold the tissue sample during retrieval and large portions of the jaws 206, 208 are coated with a non-conductive material to form non-conductive surfaces 220 that extend over the internal portions of the jaws 206, 208 to protect the sample tissue.

In this embodiment, the conductive surfaces 222 of the device are limited to the distal tip of the jaws 206, 208. For example, the outer distal ends of the forceps 202 are left uncoated to form a substantially circular cauterization zone. According to this embodiment, the shape of the conductive surfaces 222 directs RF energy toward a grounding pad and away from the sample of tissue contained within the sample chamber 216. In other exemplary embodiments, the conductive surfaces 222 may be located on external surfaces of sidewalls of the jaws 206, 208, e.g., about 180° apart. For example, the conductive surfaces 222 may be located on opposite sides of the jaws 206, 208 adjacent the contacting edges and/or on upper external portion of the jaw 206 and a lower external portion of the jaw 208 when the contacting edges are in a horizontal plane. In this manner, the forceps 202 need only be rotated, at most, about 90° to align one of the conductive surfaces 222 with the target tissue region to be cauterized. In another exemplary embodiment, the conducting surfaces 222 may be selectively activated, e.g., one on/one off. Preferably, the conductive surfaces 222 are semi-circular, forming a substantially circular shape when the jaws 206, 208 are closed.

The preceding embodiments of the biopsy forceps may be based on existing forceps used for sample tissue retrieval. For example, the Barracuda and the Tiger Shark lines of biopsy forceps, and the Stone Retrieval Devices Basket line manufactured by the Boston Scientific Corporation of Natick, Mass., may be modified to operate in accord with the teachings of the present invention by applying non-conductive coatings in to selected areas as described above.

In alternate embodiments of the present invention, the jaws or cutting elements of the device are not used to perform the cauterization of the sampling area. Instead, a separate media is used to cauterize the wound, for example by directing RF or other energy to the target tissue. According to these embodiments, a path of least resistance is provided toward the tissue to be cauterized (e.g., toward a grounding pad located on an opposite side of the tissue to be cauterized) and away from the sample tissue held within the device. The cauterization media may be a fixed or a movable protrusion that extends from the biopsy forceps towards the target tissue.

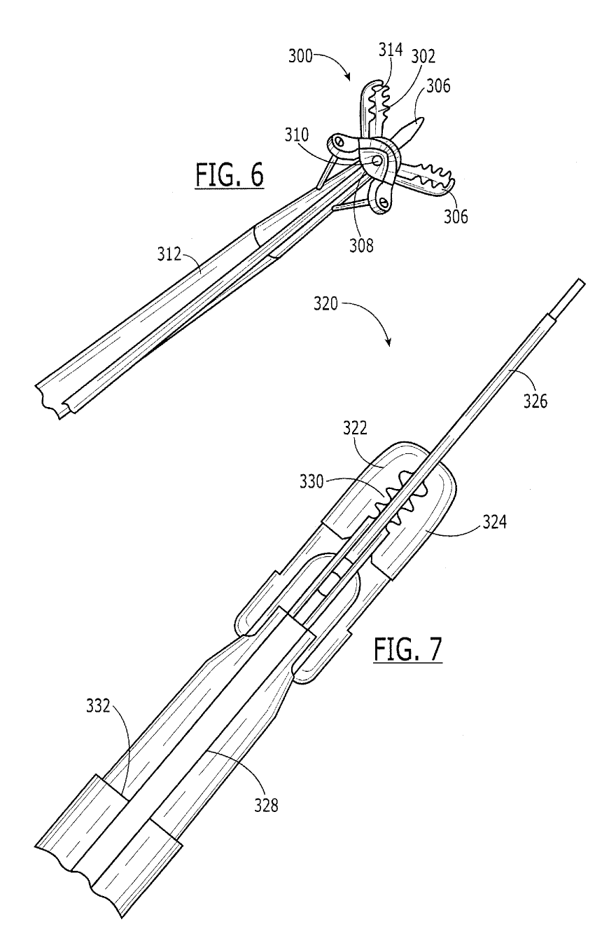

As shown in FIG. 6, forceps 300 according to an exemplary embodiment of the invention comprises a pair of jaws 302, 304 at the distal end of a shaft 312 of the forceps 300 forming a sample chamber 314 when pivoted about the hinge 310 to the closed configuration. The forceps 300 may further include a tissue grabber 306 which telescopes distally from a lumen 308 of the shaft 312 to hold tissue in place. The forceps 300 further includes an optic fiber 326 (shown in FIG. 7) which, during insertion of the device and excision of the tissue, is preferably stored within the shaft 312 and, after the sample has been placed within the sample chamber 314, extended to the site from which the tissue was removed to cauterize the resulting wound. Alternatively, the optic fiber 326 may be fixed in an extended position and activated only after the tissue sample has been enclosed within the sample chamber 314.

A biopsy forceps 320 as shown in FIG. 7, comprises a pair of pivoting jaws 322, 324 that include cutting elements to remove the sample tissue from the sampling site. As described above in regard to the previous embodiments, the jaws 322, 324 close to form a sample chamber 330 in which the tissue sample is stored for removal from the body. The laser light is transmitted to the distal end of the forceps 320 via the optic fiber 326 to a selected position for irradiating tissue in a desired spatial relation to the distal end of the forceps 320. As would be understood by those skilled in the art, heat transferred to the sample within the sample chamber 330 as the laser energy passes by via the optic fiber is minimal and only a small amount of heat is transferred to non-targeted surrounding tissue on which the laser is not aimed.

Furthermore as would be understood by those skilled in the art, other mechanisms and/or forms of energy may be used to cauterize the tissue. For example, a resistance type heater may be utilized instead of the laser and RF energy systems described above. Alternatively, a dual lumen may be provided in the shaft of the biopsy forceps according to the invention through which a wire loop (e.g., of nickel-chrome wire) for RF or other forms of electric energy may be advanced to the sampling site to cauterize the tissue. As would be understood, such devices may be powered by an external battery, RF generator or other power source and, as in the embodiments described above, the loops may be fixed or may be telescoping from the shaft of the device.

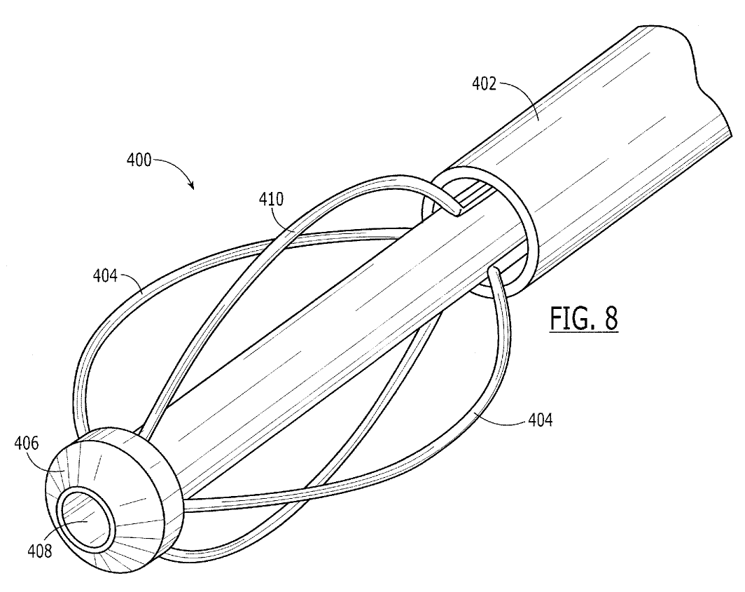

As shown in FIG. 8, a biopsy forceps 400 according to a further embodiment of the invention includes a basket 410 which captures the tissue sample after it has been excised. The basket 410 of the forceps 400 comprises a channel 408 opening at the distal end 406 and accommodating a heating element such as an RF wire, a fiber-optic cable for delivering laser energy, a nickel-chrome wire or other device which, when passed through the channel 408 to a location adjacent to the target site, may be energized to cauterize the tissue surrounding the site from which the tissue sample was removed. As described above, for electric and RF cauterizing systems, outer surfaces of the channel 408 are preferably made non-conductive, and may be used as an insulator. Teflon PTFE, Teflon PFA, ceramic or other heat resistant materials may be used to form the coating. Of course, such coatings need not be electrically non-conductive for laser based systems.

In another embodiment, the basket 410 is formed of a plurality of filaments 404 with one or more of the filaments 404 transmitting energy to the target tissue. For example, the one or more filaments 404 may be formed of an electrically conductive wire or fiber-optic cable to transmit RF, laser or other energy to a cauterization element disposed at the distal tip 406. For example, where one of the filaments 404 is used as a hot element for cauterization, the location of this filament 404 with respect to the interior of the basket 410 is selected to minimize the affect of the heat on a tissue sample retained within the basket 410. In addition, selected portions of the filament 404 may be insulated (thermally and/or electrically) to maximize energy transfer to the target tissue while minimizing energy transfer to sample tissue retained within the basket 410.

The present invention has been described with reference to specific exemplary embodiments. Those skilled in the art will understand that changes may be made in details, particularly in matters of shape, size, material and arrangement of parts. Accordingly, various modifications and changes may be made to the embodiments. The specifications and drawings are, therefore, to be regarded in an illustrative rather than a restrictive sense.

What is claimed is:

1. A biopsy device, comprising:
a tissue cutting arrangement operable to excise a tissue sample from a sampling site, the tissue cutting arrangement defining a sample chamber therewithin for receiving a tissue sample after excision by the tissue cutting arrangement, the sample chamber including insulated sample contacting surfaces; and
a cauterizing element formed on at least a portion of an exterior surface of the tissue cutting arrangement, the cauterizing element being actuatable to cauterize the sampling site after excision of the tissue sample.

2. The biopsy device of claim 1, wherein at least a portion of the tissue cutting arrangement is formed of an electrically conductive material, a first portion of the exterior surface of the tissue cutting arrangement forming the tissue cauterizing element being substantially free electrically insulating material and a second portion of the exterior surface of the tissue cutting arrangement not forming the tissue cauterizing element being covered in an electrically insulative material.

3. The biopsy device of claim 1, wherein the tissue cutting arrangement comprises hinged jaws, inner surfaces of the hinged jaws forming the sample chamber being coated with an insulating material.

4. The biopsy device of claim 3, wherein selected portions of outer surfaces of the hinged jaws are uninsulated.

5. The biopsy device of claim 4, wherein, when the hinged jaws are in a closed configuration, the selected portions of the outer surfaces form a substantially circular electrically conductive region.

6. The biopsy device of claim 4, wherein the selected portions of the outer surfaces comprise substantially the entirety of the outer surfaces of the jaws.

7. The biopsy device of claim 1, wherein the insulated sample contacting surfaces are coated with a heat insulating material.

8. The biopsy device of claim 1, wherein the insulated sample contacting surfaces are coated with one of PTFE, PFA and ceramic.

9. The biopsy device of claim 1, wherein the insulated sample contacting surfaces are electrically insulated.

10. The biopsy device of claim 1, further comprising:
an electrically conductive element extending proximally from the cauterizing element to a source of electric energy.

11. The biopsy device of claim 10, wherein the conductive element telescopes from a lumen of the tissue cutting arrangement, the lumen insulating an interior of the sample chamber from the conductive element.

12. The biopsy device of claim 1, wherein the cauterizing element comprises an optic fiber connected to a laser.

13. The biopsy device of claim 1, wherein the optic fiber telescopes from a lumen of the tissue cutting arrangement.

14. The biopsy device of claim 1, wherein the sample chamber comprises a basket formed of a plurality of filaments. T

15. The biopsy device of claim 1, wherein a first one of the filaments forming the sample chamber is formed of an electrically conductive material to serve as the cauterizing element.

16. A method of biopsy, comprising:
advancing to a sampling site within a body lumen an insulated biopsy forceps;
actuating from a cutting element of the forceps from outside the body to excise a tissue sample from the sampling site;
retaining the tissue sample in a sample chamber within the forceps, the sample chamber having insulated interior surfaces; and
energizing a cauterizing element to cauterize the sampling site.

17. The method of claim 16, wherein the cutting element includes opposed jaws and wherein actuating the cutting element pivots the jaws relative to one another.

18. The method of claim 17, wherein the non-insulated portion of the forceps comprises an electrically conductive portion of outer surfaces of the jaws and wherein the non-insulated portion of the forceps is energized by supplying electric energy thereto.

19. The method of claim 18, wherein the electrically conductive portion of the outer surfaces is substantially circular when the jaws are pivoted into contact with one another.

20. The method of claim 17, wherein the sample chamber is defined by interior surfaces of the jaws.

21. The method of claim 16, further comprising:
extending the cauterizing element from the forceps to contact the sampling site.

22. The method of claim 21, wherein the cauterizing element is advanced by telescoping out of the forceps.

23. The method of claim 21, wherein the cauterizing element is coupled to a source of electrical energy.

24. The method of claim 24, wherein the electrical energy is RF energy.

25. The method of claim 21, wherein the cauterizing element comprises an optic fiber coupled to a source of laser energy.

26. The method of claim 17, further comprising:
extending the optic fiber distally of the forceps toward the sampling site.

27. The method of claim 17, wherein the tissue sample is a basket formed of multiple filaments.

28. The method of claim 21, wherein the cauterizing element includes a first one of the filaments.

29. A biopsy system with cauterization, comprising:
a shaft having a distal end insertable through a scope to a sampling site within a body;
a non-conductive shaft coating covering at least a portion of the shaft;
a housing disposed at a distal end of the shaft;
a cutting element of the housing operable by a user to excise a sample tissue from the sampling site;
a sample chamber defined within the housing for retaining the excised sample tissue;
a cauterization element of the housing to cauterize the sampling site after excision of the sample tissue; and
a non-conductive housing coating covering at least parts of surfaces of the housing.

30. The biopsy system wherein the cutting element comprises jaws pivotable between an open and a closed position.

31. The biopsy system of claim 30, wherein the sample chamber is formed by non-conductive forceps cups defined within the jaws.

32. The biopsy system of claim 30, wherein the cauterization element comprises a non-coated portion of the cutting element.

33. The biopsy system of claim 31, wherein the cauterization element comprises a non-coated portion of the jaws.

34. The biopsy system of claim 31, wherein the non-coated portion is substantially circular when the jaws are closed.

35. The biopsy system of claim 29, wherein the cauterization element comprises a heated conductive element extending from the housing.

36. The biopsy system of claim 29, wherein the cauterization element comprises a source of laser energy coupled to an optic fiber extendable distally from the housing.

37. The biopsy system of claim 29, wherein the shaft coating and the housing coating comprise at least one of PTFE, PFA and ceramic material.
